(19) United States
(12) Patent Application Publication
(10) Pub. No.: US 2026/0031193 A1
(43) Pub. Date: Jan. 29, 2026

(54) ANALYZING COLORIMETRIC TEST RESULTS OF DRILLING MUD USING COMPUTER VISION

(71) Applicant: Halliburton Energy Services, Inc., Houston, TX (US)

(72) Inventors: Mateusz Michal DYNGOSZ, Houston, TX (US); Dale E. JAMISON, Houston, TX (US)

(73) Assignee: Halliburton Energy Services, Inc., Houston, TX (US)

(21) Appl. No.: 18/786,984

(22) Filed: Jul. 29, 2024

(51) Int. Cl. G16C 20/30; G01N 21/78; G01N 33/28; G16C 20/70

(52) U.S. Cl. CPC G16C 20/30; G01N 21/78; G01N 33/2823; G16C 20/70

(57) ABSTRACT

Systems and methods are provided for evaluation of the cement bonding condition in a wellbore based on borehole resonance mode using machine learning. An example method can include transforming the return signal into a resonance signal based on feature extraction of the return signal, determining a segment of the resonance signal in a time domain, and determining, via a machine learning model, a predicted borehole cement bonding based on the segment of the resonance signal. The example method can further include generating a bonding log based on the predicted borehole cement bonding.

### TECHNICAL FIELD

The present disclosure generally relates to analyzing colorimetric testing of drilling fluid using machine learning. For example, aspects of the present disclosure relate to analyzing colorimetric test results of drilling fluid (e.g., drilling mud) using a neural network.

### BACKGROUND

Wells can be drilled to access and produce hydrocarbons such as oil and gas from subterranean geological formations. Wellbore operations can include drilling operations, completion operations, fracturing operations, and production operations. Drilling operations may involve gathering information related to downhole geological formations of the wellbore, conditions of the drilling fluid, and the drilling fluid's compatibility with the formation being drilled. The information may be collected by wireline logging, logging while drilling (LWD), measurement while drilling (MWD), drill pipe conveyed logging, coil tubing conveyed logging, or surface measurements.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only exemplary embodiments of the disclosure and are not, therefore, to be considered to be limiting of its scope, the principles herein are described and explained with additional specificity and detail through the use of the accompanying drawings in which:

FIG. 1A is a schematic side-view of an example logging while drilling environment, according to some examples of the present disclosure.

FIG. 1B is a schematic diagram of an example downhole environment having tubulars, according to some examples of the present disclosure.

FIG. 2 illustrates an example system for analyzing colorimetric test results of drilling fluid, in accordance with aspects of the present disclosure.

FIG. 3A illustrates an example system including a camera and a computing device for processing an image of a colorimetric testing result of drilling fluid, according to some aspects of the disclosed technology.

FIG. 3B illustrates an example automated system for preparing a colorimetric test for drilling fluid and processing an image of the test result, according to some aspects of the disclosed technology.

FIG. 4 illustrates an example automated system of preparing another colorimetric test for drilling fluid, according to some aspects of the disclosed technology.

FIG. 5 illustrates a flowchart of an example process for analyzing colorimetric test results of drilling fluid using machine learning, according to some aspects of the disclosed technology.

FIG. 6 illustrates an example neural network, according to some aspects of the disclosed technology.

FIG. 7 illustrates an example computing device architecture which can be employed to perform various steps, methods, and techniques disclosed herein.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

Additional features and advantages of the disclosure will be set forth in the description which follows, and in part will be obvious from the description, or can be learned by practice of the principles disclosed herein. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Drilling fluids (also referred to as drilling mud) are used in various stages and applications within the drilling process. Drilling fluids are crucial in maintaining the stability of the wellbore walls during the drilling process, preventing collapse and ensuring the integrity of the borehole. As follows, during a drilling operation, for example in the oil and gas industry, drilling fluids are controlled, measured, and monitored to ensure well control and pressure management, formation protection, optimizing drilling performance, and so on. However, the lithology and active shale content of the formation are not always accurately known prior to drilling and can be different than what is expected or planned.

Further, different compositions of drilling fluids can be tailored to achieve specific properties that enhance drilling performance under varying operating conditions in a well. Drilling fluids can be tested to measure and control their properties, ensuring the fluid meets the specific requirements of the drilling operation such that testing and monitoring can allow for timely adjustments to maintain optimal performance and address any issues that arise during drilling. Some colorimetric tests (e.g., methylene blue test (MBT), alkalinity test, etc.) can be used to determine the chemical properties of drilling fluids, for example, by examining the color-based measurements. However, human visual assessment for colorimetric tests can lack precision, accuracy, reliability, and consistency as different individuals may perceive colors differently due to variations in color vision and interpretation. Also, the same person may interpret colors differently under different conditions or over time.

Systems, apparatuses, processes (also referred to as methods), and computer-readable media (collectively referred to as “systems and techniques” or “system”) are described herein for analyzing colorimetric test results of drilling fluid using machine learning (e.g., neural network). For example, the systems and techniques of the present disclosure can receive image data that captures a result of a colorimetric testing of a drilling fluid and apply a machine learning method for analyzing the image data to determine one or more properties of the drilling fluid.

Further, the systems and techniques of the present disclosure can determine, in response to determining that at least one of the properties of the drilling fluid exceeds a predetermined threshold, a remedial action to adjust parameter(s) of a drilling fluid treatment schedule. In some examples, a change in drilling rate of penetration (ROP), weight on bit (WOB), mud weight, etc. may be prescribed either temporarily or for continuing operations, due to the time required to implement a chemical change to the mud system.

In some examples, the techniques and technologies described herein can analyze the colorimetric test results of drilling fluid using image processing techniques. In some aspects, the systems and techniques of the present disclosure can deploy a set of neural networks that are specifically trained for different property detections. In some implementations, the systems and techniques of the present disclosure can use a combination of an image processing technology and neural network(s).

In some examples, managing a colorimetric test with a sample of drilling fluid and analyzing the test result can be automated with minimal human input and performed in real time. As discussed in further detail below, the technologies and techniques described herein can improve the safety, efficiency, and cost-effectiveness of drilling operations, in particular, drilling fluid treatment by providing solutions for analyzing colorimetric test results of drilling mud using computer vision (e.g., deep neural network) and optimizing a drilling fluid treatment plan/schedule. Further, the technologies and techniques described herein can help determine the formation and various formation details (e.g., organic materials, etc.), which can be used to determine a mud system treatment prior to casing.

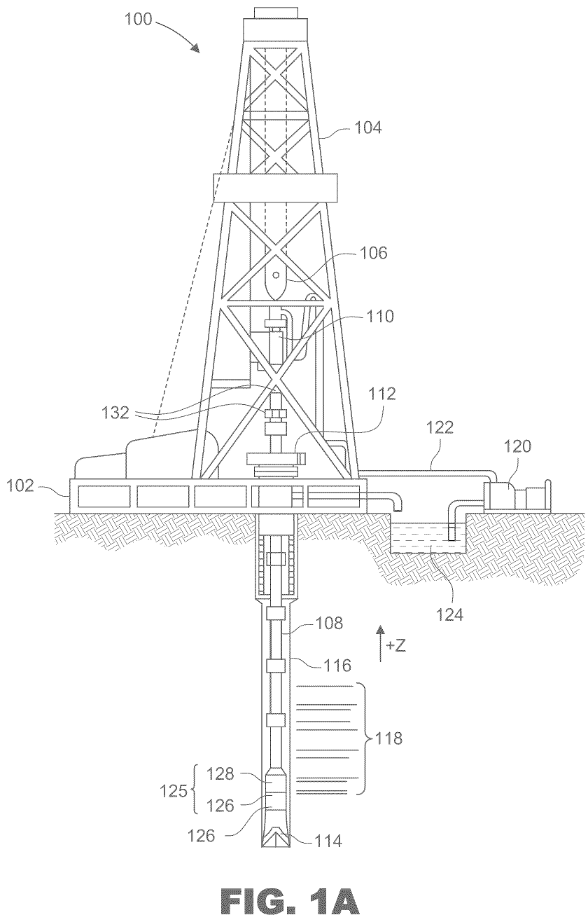

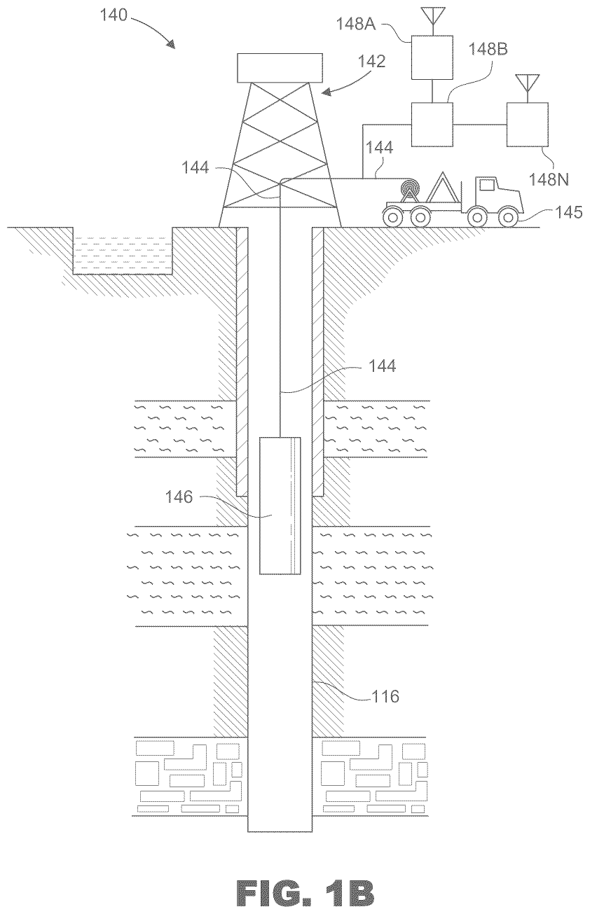

Examples of the systems and techniques described herein are illustrated in FIG. 1A through FIG. 7 and described below.

Turning now to FIG. 1A, a drilling arrangement is shown that exemplifies a Logging While Drilling (commonly abbreviated as LWD) configuration in a wellbore drilling scenario 100. Logging-While-Drilling typically incorporates sensors that acquire formation data. Specifically, the drilling arrangement shown in FIG. 1A can be used to gather formation data through an electromagnetic imager tool as part of logging the wellbore using the electromagnetic imager tool. The drilling arrangement of FIG. 1A also exemplifies what is referred to as Measurement While Drilling (commonly abbreviated as MWD) which utilizes sensors to acquire data from which the wellbore's path and position in three-dimensional space can be determined. FIG. 1A shows a drilling platform 102 equipped with a derrick 104 that supports a hoist 106 for raising and lowering a drill string 108. The hoist 106 suspends a top drive 110 suitable for rotating and lowering the drill string 108 through a well head 112. A drill bit 114 can be connected to the lower end of the drill string 108. As the drill bit 114 rotates, it creates a wellbore 116 that passes through various subterranean formations 118. A pump 120 circulates drilling fluid through a supply pipe 122 to top drive 110, down through the interior of drill string 108 and out orifices in drill bit 114 into the wellbore. The drilling fluid returns to the surface via the annulus around drill string 108, and into a retention pit 124. The drilling fluid transports cuttings from the wellbore 116 into the retention pit 124 and the drilling fluid's presence in the annulus aids in maintaining the integrity of the wellbore 116. Various materials can be used for drilling fluid, including oil-based fluids and water-based fluids.

Logging tools 126 can be integrated into the bottom-hole assembly 125 near the drill bit 114. As the drill bit 114 extends into the wellbore 116 through the formations 118 and as the drill string 108 is pulled out of the wellbore 116, logging tools 126 collect measurements relating to various formation properties as well as the orientation of the tool and various other drilling conditions. The logging tool 126 can be applicable tools for collecting measurements in a drilling scenario, such as the electromagnetic imager tools described herein. Each of the logging tools 126 may include one or more tool components spaced apart from each other and communicatively coupled by one or more wires and/or other communication arrangement. The logging tools 126 may also include one or more computing devices communicatively coupled with one or more of the tool components. The one or more computing devices may be configured to control or monitor a performance of the tool, process logging data, and/or carry out one or more aspects of the methods and processes of the present disclosure.

The bottom-hole assembly 125 may also include a telemetry sub 128 to transfer measurement data to a surface receiver 132 and to receive commands from the surface. In at least some cases, the telemetry sub 128 communicates with a surface receiver 132 by wireless signal transmission (e.g., using mud pulse telemetry, EM telemetry, or acoustic telemetry). In other cases, one or more of the logging tools 126 may communicate with a surface receiver 132 by a wire, such as wired drill pipe. In some instances, the telemetry sub 128 does not communicate with the surface, but rather stores logging data for later retrieval at the surface when the logging assembly is recovered. In at least some cases, one or more of the logging tools 126 may receive electrical power from a wire that extends to the surface, including wires extending through a wired drill pipe. In other cases, power is provided from one or more batteries or via power generated downhole.

Collar 134 is a frequent component of a drill string 108 and generally resembles a very thick-walled cylindrical pipe, typically with threaded ends and a hollow core for the conveyance of drilling fluid. Multiple collars 134 can be included in the drill string 108 and are constructed and intended to be heavy to apply weight on the drill bit 114 to assist the drilling process. Because of the thickness of the collar's wall, pocket-type cutouts or other type recesses can be provided into the collar's wall without negatively impacting the integrity (strength, rigidity and the like) of the collar as a component of the drill string 108.

Referring to FIG. 1B, an example system 140 is depicted for conducting downhole measurements after at least a portion of a wellbore has been drilled and the drill string removed from the well. A downhole tool can be operated in the example system 140 shown in FIG. 1B to log the wellbore. A downhole tool is shown having a tool body 146 in order to carry out logging and/or other operations. For example, instead of using the drill string 108 of FIG. 1A to lower the downhole tool, which can contain sensors and/or other instrumentation for detecting and logging nearby characteristics and conditions of the wellbore 116 and surrounding formations, a wireline conveyance 144 can be used. The tool body 146 can be lowered into the wellbore 116 by wireline conveyance 144. The wireline conveyance 144 can be anchored in the drill rig 142 or by a portable means such as a truck 145. The wireline conveyance 144 can include one or more wires, slicklines, cables, and/or the like, as well as tubular conveyances such as coiled tubing, joint tubing, or other tubulars. The downhole tool can include an applicable tool for collecting measurements in a drilling scenario, such as the electromagnetic imager tools described herein.

The illustrated wireline conveyance 144 provides power and support for the tool, as well as enabling communication between data processors 148A-N on the surface. In some examples, the wireline conveyance 144 can include electrical and/or fiber optic cabling for carrying out communications. The wireline conveyance 144 is sufficiently strong and flexible to tether the tool body 146 through the wellbore 116, while also permitting communication through the wireline conveyance 144 to one or more of the processors 148A-N, which can include local and/or remote processors. The processors 148A-N can be integrated as part of an applicable computing system, such as the computing device architectures described herein. Moreover, power can be supplied via the wireline conveyance 144 to meet power requirements of the tool. For slickline or coiled tubing configurations, power can be supplied downhole with a battery or via a downhole generator.

Although FIGS. 1A and 1B depict specific borehole configurations, it should be understood that the present disclosure is suited for use in wellbores having other orientations including vertical wellbores, horizontal wellbores, slanted wellbores, multilateral wellbores, and the like. While FIGS. 1A and 1B depict an onshore operation, it should also be understood that the present disclosure is suited for use in offshore operations. Moreover, the present disclosure is not limited to the environments depicted in FIGS. 1A and 1B, and can also be used in other well operations such as, for example and without limitation, production tubing operations, jointed tubing operations, coiled tubing operations, combinations thereof, and/or the like.

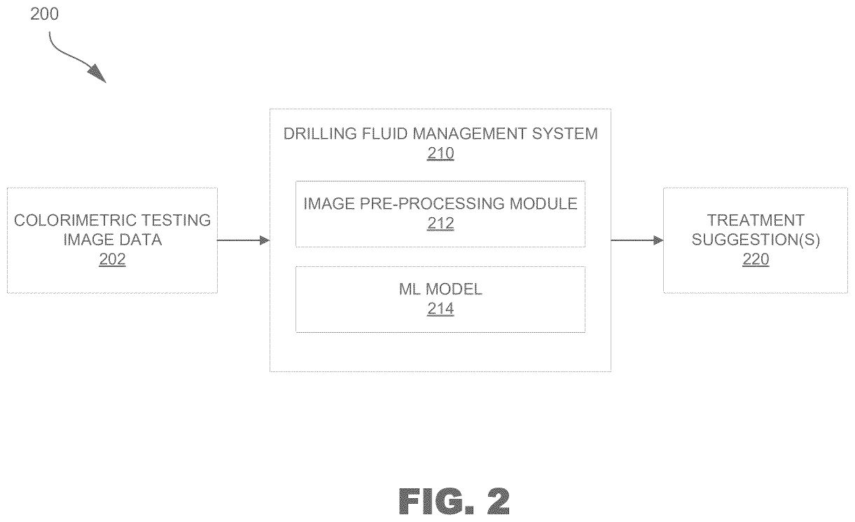

FIG. 2 illustrates an example system 200 for analyzing colorimetric test results of drilling fluid. As shown, the system 200 includes drilling fluid management system 210, which is configured to receive colorimetric testing image data 202 and generate treatment suggestion(s) 220. For example, drilling fluid management system 210 is configured to perform applicable functions related to analyzing colorimetric testing image data 202 to identify one or more color metric measurements, which may indicate properties of drilling fluid (e.g., drilling fluid circulated through pump 120 as illustrated in FIGS. 1A and 1B). In some aspects, example system 200 can be implemented in the one or more computing devices, as illustrated in FIG. 1A, which is configured to control or monitor a performance of the tool (e.g., logging tool 126) and process logging data such that the combination of measurements collected by the tool and analysis of the test results can improve the drilling operation.

The colorimetric testing image data 202 can be obtained from various colorimetric tests such as a methylene blue test (MBT), an alkalinity test for Pf, Mf, and Pm, and so on. The colorimetric testing image data 202 may include a collection of images that capture the result of the test. For a methylene blue test, colorimetric testing image data 202 includes an image of droplet(s) of the mixture of drilling fluid and methylene blue solution at various concentrations. For an alkalinity test, colorimetric testing image data 202 includes multiple images that are captured continuously over a period of time to show the progress of a color change over time.

In some implementations, drilling fluid management system 210 can include image pre-processing module 212, which functions to pre-process colorimetric testing image data 202. For example, drilling fluid management system 210 can adjust the resolution of an image included in the colorimetric testing image data 202, build the image into grayscale, change the contract, and so on to optimize the image quality for subsequent processing. Non-limiting examples of image pre-processing techniques include color space transform/conversion (e.g., RGB to HSV, Lab, YCbCr, etc.), thresholding, color histograms, pixel-wise color analysis, edge detection in color spaces, color segmentation, K-means clustering for color quantization, Gaussian mixture models for color modeling, a watershed algorithm for color-based segmentation, blob detection in color images, template matching for specific color patterns, Hough transformation for detection and color shapes, chroma keying (e.g., green screen technique), color moment analysis, and color correlogram.

The drilling fluid management system 210 can use an algorithm, such as a machine learning algorithm, to analyze colorimetric testing image data 202. For example, drilling fluid management system 210 can include an applicable machine learning-based technique or neural network, which is configured to identify color metric measurements (e.g., color change that appears in image data 202, etc.) and determine one or more properties of the drilling fluid based on the color metric measurements. As such, drilling fluid management system 210 can generate treatment suggestion(s) 220, in a given well, to adjust the drilling fluid treatment schedule. Non-limiting examples of ML model 214 (e.g., neural network) can include a deep neural network (DNN), convolutional neural network (CNN), Convolutional Long Short-Term Memory (ConvLSTM), Vision Transformer (for time-series image processing), hidden Markov models, Recurrent Neural Network (RNN), deep learning, and Generative Adversarial Network (GAN), among others.

In some aspects, drilling fluid management system 210 can access additional data associated with test conditions or drilling fluid used in a test. For example, ML model 214 can be fed with testing conditions (e.g., light intensity, test products, etc.), information about the clay derived from the given well, historical test data, and so on.

In some implementations, drilling fluid management system 210 can generate treatment suggestion(s) 220 based on processing and analysis of colorimetric test image data 202. The treatment suggestions 220 can include, for example, adjusting one or more parameters of the drilling fluid treatment schedule or plan such as a rate of fluid treatment, a drilling fluid inhibition factor, a salt concentration of the aqueous drilling fluid, a rate of penetration (ROP), bit RPM, bit type, weight on bit (WOB), pump rate, or a combination thereof. In some examples, treatment suggestions 220 can include adjusting or changing treatment methods based on a determination of depletion of the chemicals in the mud. In some aspects, treatment suggestions 220 can include changes to the future drilling programs based on the changes to the mud registered by the system. In some cases, treatment suggestions 220 can include suggestions of different chemicals that may be used in the fluid if the current chemicals are not sufficiently effective. Further, treatment suggestions 220 can include adjusting, for water base mud, shale swelling inhibitors or other chemical additions, salt content, lubricant content, mud weight, pH, and so on. For oil mud, treatment suggestions 220 can include adjusting, water phase salinity of the internal phase, emulsifier concentration, other treatment chemicals, mud weight, etc. In some examples, drilling fluid management system 210 can communicate with (or transmit data to) surface receiver 132 as illustrated in FIG. 1A such that the data analyzed from test results of the drilling fluid or treatment suggestions 220 can be used along with measurement data from logging tool to improve the drilling operation.

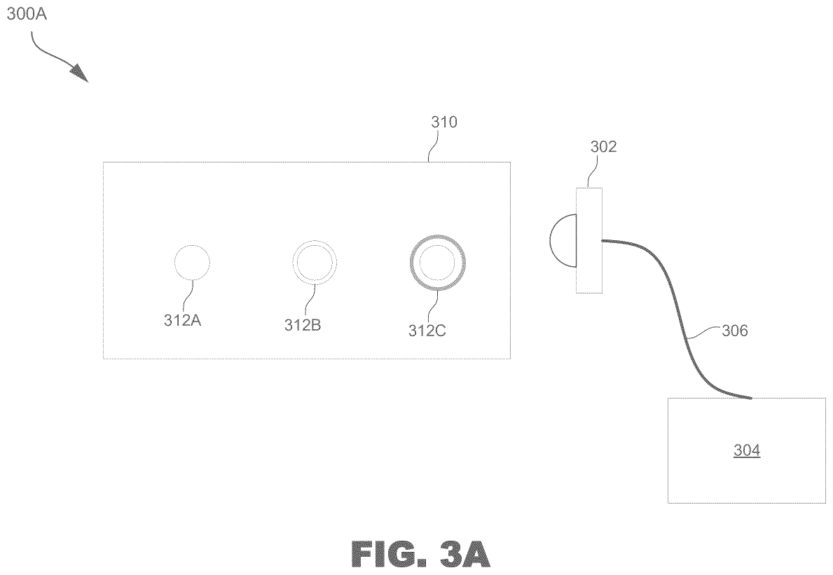

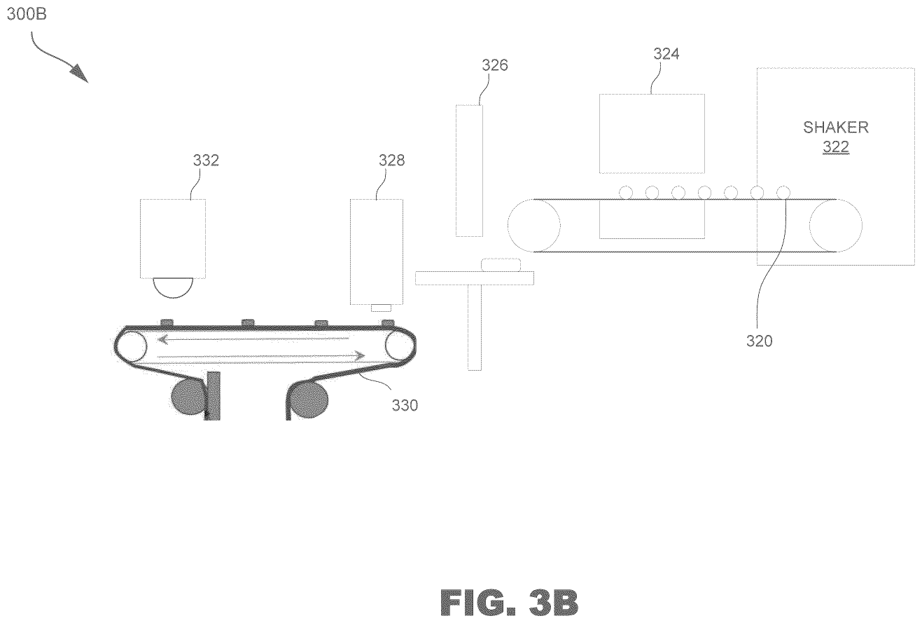

FIG. 3A illustrates an example system 300A including a camera 302 and a computing device 304 (e.g., System on Chip (SoC)) for processing an image 310 of a colorimetric testing result of drilling fluid, according to some aspects of the disclosed technology. For example, camera 302 is coupled to computing device 304 via a cable 306 (e.g., ribbon or USB cable). In some examples, computing device 304 can be equipped with drilling fluid management system 210 as illustrated in FIG. 2, which is configured to process and analyze colorimetric test results of drilling fluid and generate or update a drilling fluid treatment schedule. In some examples, image data captured by camera 302 can be transmitted to computing device 304 remotely/wirelessly without cable 306.

In some examples, camera 302 can capture image 310 (similar to colorimetric testing image data 202 as illustrated in FIG. 2), which shows droplets 312A, 312B, 312C from a methylene blue test (MBT). For example, a methylene blue dye solution can be added to a sample of drilling fluid. After adding each drop of methylene blue solution, the mixture of the solution and drilling fluid can be placed, as a drop, on a filter paper. For example, droplet 312A is when 4 mL of methylene blue solution is added, droplet 312B is when 5 mL of methylene blue solution is added, and droplet 312C is when 6 mL of methylene blue solution is added. The dye solution can continue to be incrementally added to the mixture until all the reactive clay surfaces in the drilling fluid are saturated.

The camera 302 can capture image 310 of droplets 312A-C, which then can be transmitted to computing device 304 for processing. The computing device 304 (e.g., computer vision system) can analyze image 310 to determine a color of each droplet, a color gradient, a color change over time between droplets, a shape of each droplet, a shape and/or size of a permanent blue halo around the central spot, and so on.

In some aspects, computing device 304 can monitor the change between droplets over time. If the change satisfies predetermined parameters, computing device 304 can transmit a notification to a user to notify the change.

A machine learning algorithm (e.g., ML model 214) can be embedded on computing device 304 such that a neural network or image processing algorithm can be used to detect the colors, edges, color gradients, and shapes associated with droplets 312A, 312B, 312C on image 310.

In some implementations, computing device 304 can determine, based on the analysis of image 310, one or more characteristics of the drilling fluid. For example, the MBT result can provide cation exchange capacity of the clay solids in the drilling fluid. For example, ML model 214 may analyze the methylene blue reaction with time and color development. Further, based on the analysis of image 310, ML model 214 can make predictions as to shale activity and clay types present in the formation (e.g., formation 118). The ML model 214 can also make clay activity predictions ahead of the bit based on the trending data. As follows, computing device 304 can create and design a drilling fluid treatment schedule (also referred to as a treatment plan) taking into account the characteristics of the drilling fluid.

In some examples, the evaluation or analysis of image 310 can be presented to a user on a screen or display device that is associated with the user. For example, computing device 304 can transmit, over a network, results of the test, analysis of the test results, and predictions based on the analysis to a user. Further, computing device 304 can annotate image 310 on the screen to add notes, labels, or other types of information onto the image 310 to provide additional context, explanations, or highlights.

FIG. 3B illustrates an example automated system 300B for preparing a colorimetric test for drilling fluid and processing an image of the test result, according to some aspects of the disclosed technology. As shown, cuttings 320 (e.g., mud solids) can be provided or dispensed from shale shaker 322. The crusher 324 then grinds and processes cuttings 320. In some examples, instead of cuttings, water-based mud solids can be used in the same manner. The weighing device 326 can determine the weight of cuttings 320 so that how much treatment fluid is to be added can be determined. The treatment fluid provider 328 adds treatment fluid to cuttings 320 that are placed on a reel 330 loaded with a roll of filter paper. The reel 330 allows multiple tests in succession without having to place individual pieces of paper for each test. The cuttings 320 that are added with various concentrations of methylene blue solution can be rolled over to the bottom of camera 332. The camera 332 captures image of the cuttings 320 mixed with the treatment fluid.

In some implementations, camera 332 (similar to camera 302 as illustrated in FIG. 3A) can be coupled to computing device 304 or drilling fluid management system 210 such that a collection of images that is taken by camera 332 can be processed and analyzed in real time and without manual human input.

In some aspects, based on the analysis of multiple test results, drilling fluid management system 210 can determine the characteristics of the drilling fluid or cuttings 320 and therefore, make predictions as to clay types and activities. As follows, drilling fluid management system 210 can adjust one or more parameters of a drilling fluid treatment schedule in real time to optimize the drilling fluid performance.

In some examples, the frequency of the testing or a volume of the methylene blue solution can be determined and automatically controlled by drilling fluid management system 210 based on the relative difference between sequential tests.

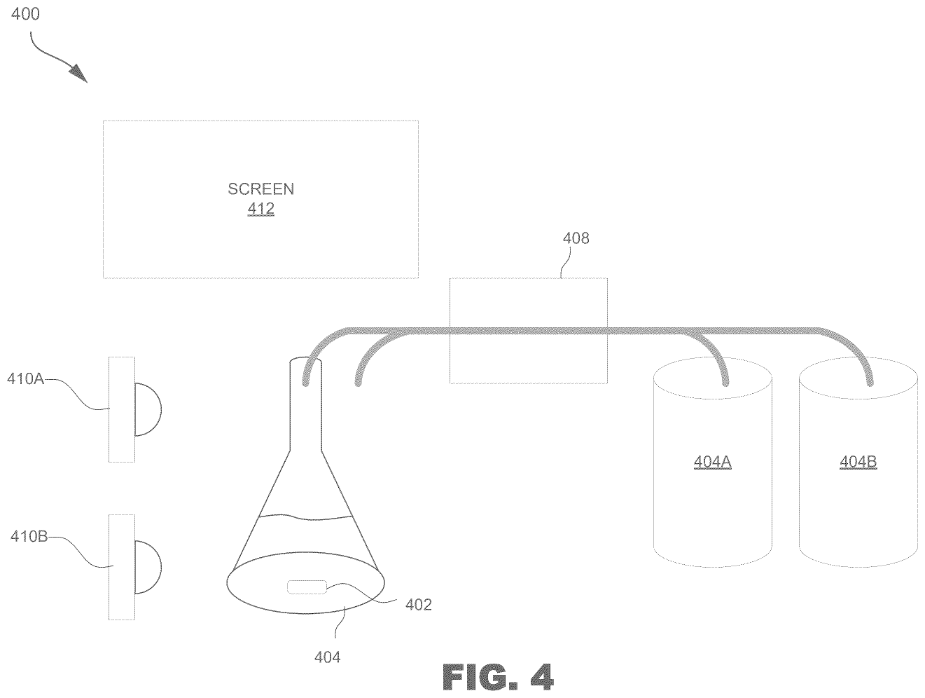

FIG. 4 illustrates an example automated system 400 of preparing a colorimetric test for drilling fluid. The automated system 400 can be set up for a colorimetric test that determines an alkalinity or acidity of drilling fluids such as Phenolphthalein Alkalinity (Pf), Methyl Orange Alkalinity (Mf), Total Alkalinity (Pm), and so on.

A sample of drilling mud 402 can be placed in flask 404. As shown, reagents 404A, 404B can be provided, via pumping/dosing system 408, to flask 404 for reaction with the sample of drilling mud 402. A set of cameras 410A, 410B can be used to capture the color development in flask 404. Further, the set of cameras 410A, 410B can be coupled to a system on chip (e.g., computing device 304 or drilling fluid management system 210). While the example automated system 400 includes a set of two cameras 410A, 410B, a single camera or any applicable number of cameras can be used without departing the scope of the present disclosure.

In some examples, drilling fluid management system 210 or ML model 214 can analyze a collection of images (e.g., continuous images or video) captured by the set of cameras 410A, 410B such as colors, color changes, optical patterns of the chemical reactions inside of flask 404, or any other parts of the chemical reaction. Based on the analysis, drilling fluid management system 210 or ML model 214 can determine drilling fluid formation, mud system interaction, impact of possible contaminations, etc. In some examples, drilling fluid management system 210 or ML model 214 can further determine a pH stability of the drilling fluids, the presence of contaminants (e.g., cement, carbonates, etc.), the effectiveness of lime or caustic soda treatments, potential corrosion in drilling fluid system, and/or overall balance of the drilling fluid in terms of acidity, bacterial growth, and so on. Further, the results of colorimetric indicators in combination of lithology knowledge, which can be gained by data from offset wells allow the fluid system composition to be managed proactively rather than reactively and therefore, help to promote trouble-free drilling without non-productive time (NPT).

In some implementations, the automated system 400 can include a built-in sequence of the events that would happen after the confirmations from ML model 214 (e.g., the computer vision algorithms) that is configured to detect desired test results.

In some examples, the start time of the test, the end time of each of the test, the progress of the test the result, or any applicable information associated with the test can be recorded and provided to the user (e.g., presented on screen 412 for user to view).

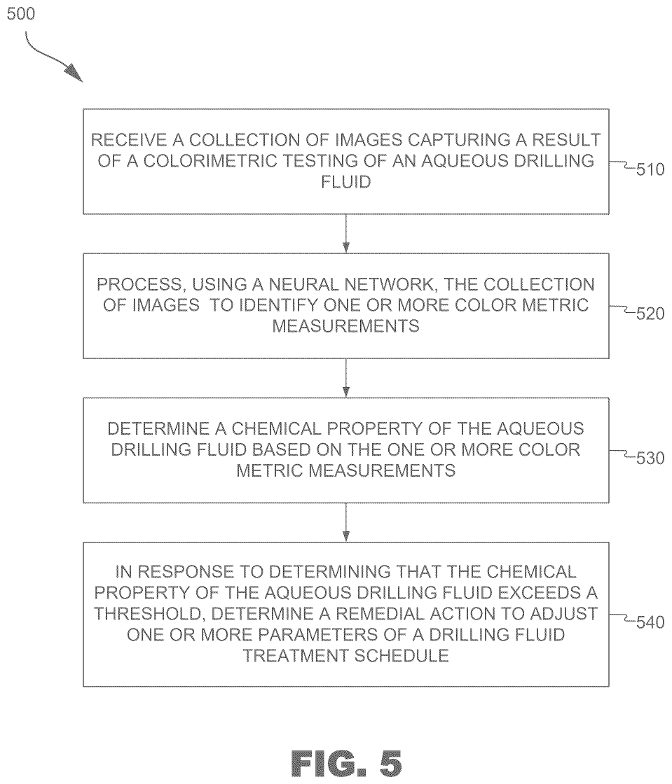

FIG. 5 illustrates a flowchart of an example process 500 for analyzing colorimetric test results of drilling fluid using machine learning. Although example process 500 depicts a particular sequence of operations, the sequence may be altered without departing from the scope of the present disclosure. For example, some of the operations depicted may be performed in parallel or in a different sequence that does not materially affect the function of process 500. In other examples, different components of an example device or system that implements process 500 may perform functions at substantially the same time or in a specific sequence.

At step 510, process 500 includes receiving a collection of images capturing a result of a colorimetric testing of an aqueous drilling fluid. For example, drilling fluid management system 210 can receive colorimetric testing image data 202, which comprises a collection of images. The collection of images can be captured by camera 302 (or camera 332, a set of cameras 410A, 410B) and represents a result of colorimetric testing of an aqueous drilling fluid.

In some implementations, the colorimetric testing can be a methylene blue test (MBT), which can be used to determine the amount of reactive clay (bentonite or montmorillonite) in drilling fluids. For example, image data from a methylene blue test (e.g., a test setup as described in FIGS. 3A and 3B) can be provided to drilling fluid management system 210. The image data from a methylene blue test can include an image of one or more droplets 312A-C of the mixture of drilling fluids and methylene blue solution on a paper.

In some examples, the colorimetric testing can be an alkalinity/acidity test, which can be used to determine Pf, Mf, Pm, and so on. For example, image data from an alkalinity test (e.g., a test setup as described in FIG. 4) can be provided to drilling fluid management system 210 for image processing. The image data from an alkalinity test comprises a collection of continuous images that are captured continuously to show the progress of the test results (e.g., a color change over time).

At step 520, process 500 includes processing, a neural network, the collection of images to identify one or more color metric measurements. For example, drilling fluid management system 210 can process and analyze, using ML model 214, colorimetric testing image data 202 to identify color metric measurements. As previously described, a machine learning algorithm (e.g., CNN, GAN, RNN, etc.) can be used to analyze the colorimetric testing image data 202.

In some implementations, processing the image of the result of the colorimetric testing of the aqueous drilling fluid includes analyzing one or more attributes associated with the one or more color metric measurements. The one or more attributes include, for example for a methylene blue test, a color, a gradient, a shape, a color change with respect to time, a shape and/or size of a permanent blue halo around the central spot, and so on.

In some examples, process 500 includes providing, to the neural network, data associated with at least one of light intensity, a density of the aqueous drilling fluid, and products used in the colorimetric testing. For example, drilling fluid management system 210 can access information associated with the testing conditions or drilling fluid conditions to analyze the image data along with the additional information.

At step 530, process 500 includes determining a chemical property of the aqueous drilling fluid based on the one or more color metric measurements. For example, drilling fluid management system 210 can determine various properties and characteristics of drilling fluid based on the analysis of colorimetric testing image data 202.

In cases where the colorimetric testing is a methylene blue test, drilling fluid management system 210 can determine various properties and characteristics of drilling fluid such as the amount of reactive lays, which can be represented with an estimate of the total cation exchange capacity (CEC) of the solids in the drilling fluid.

In cases where the colorimetric testing is an alkalinity/acidity test, drilling fluid management system 210 can determine mud alkalinity and filtrate alkalinity. In particular, drilling fluid management system 210 can determine Pf, Mf, Pm, and the amount of chemicals or possible contamination included in the drilling fluid.

At step 540, process 500 includes, in response to determining that the chemical property of the aqueous drilling fluid exceeds a threshold, determining a remedial action to adjust one or more parameters of a drilling fluid treatment schedule. For example, drilling fluid management system 210 can, based on the properties identified at step 530, determine one or more actions that can be taken to adjust one or more parameters of a drilling fluid treatment schedule such that drilling fluid performance can be optimized in drilling operations.

In some aspects, the one or more parameters of the drilling fluid treatment schedule, a rate of fluid treatment, a drilling fluid inhibition factor, a salt concentration of the aqueous drilling fluid, or a combination thereof.

Further, process 500 can include, in response to determining that the chemical property of the aqueous drilling fluid exceeds the threshold, transmitting a notification to a user to provide an analysis of the collection of images and the remedial action.

In some examples, process 500 can include in response to determining that the chemical property of the aqueous drilling fluid exceeds the threshold, generating an updated drilling fluid treatment schedule including the remedial action for a user. For example, drilling fluid management system 210 can automatically generate an updated drilling fluid treatment schedule, in response to determining that a property of the drilling fluid exceeds a predetermined threshold (e.g., threshold value, threshold range).

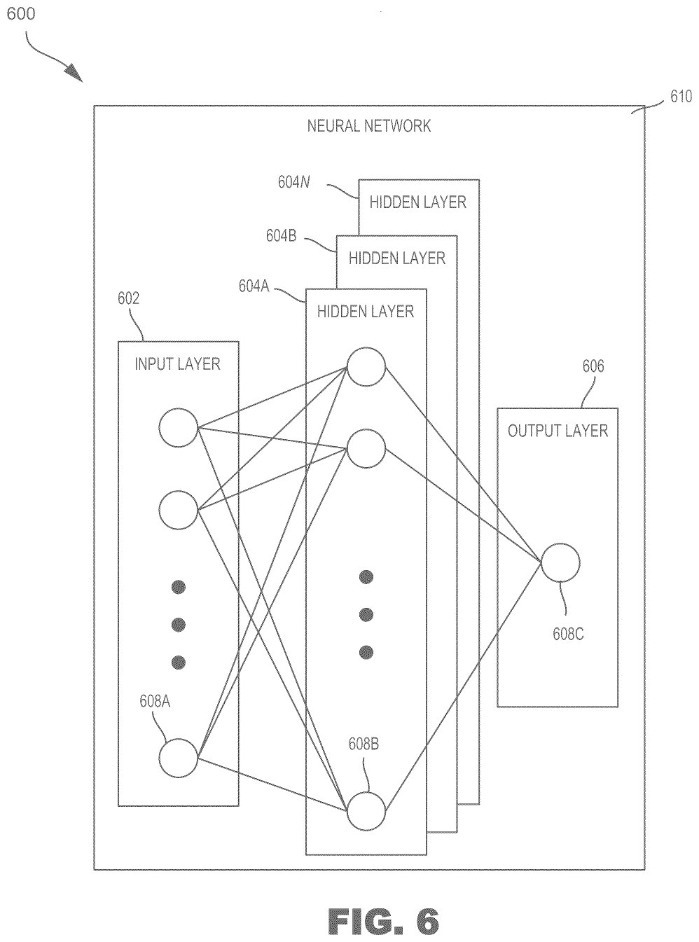

FIG. 6 illustrates an example of a neural network 610 according to some examples of the present disclosure. The neural network 610 can be used to implement any of the models described herein, such as ML model 214. As shown in this example, the neural network 610 includes an input layer 602 for processing input data. The neural network 610 also includes hidden layers 604A through 604N (collectively “604” hereinafter). The hidden layers 604 can include n number of hidden layers, where n is an integer greater than or equal to one. The number of hidden layers can include as many layers as needed for a desired processing outcome and/or rendering intent. The neural network 610 includes an output layer 606 that provides an output resulting from the processing performed by the hidden layers 604.

The neural network 610 in this example is a multi-layer neural network of interconnected nodes. Each node can represent a piece of information. Information associated with the nodes is shared among the different layers and each layer retains information as information is processed. In some cases, the neural network 610 can include a feed-forward neural network, in which case there are no feedback connections where outputs of the neural network are fed back into itself. In other cases, the neural network 610 can include a recurrent neural network, which can have loops that allow information to be carried across nodes while reading in input.

Information can be exchanged between nodes through node-to-node interconnections between the various layers. Nodes of the input layer 602 can activate a set of nodes in the first hidden layer 604A. For example, as shown, each of the input nodes of the input layer 602 is connected to each of the nodes of the first hidden layer 604A. The nodes of the hidden layer 604A can transform the information of each input node by applying activation functions to the information. The information derived from the transformation can then be passed to and can activate the nodes of the next hidden layer (e.g., 604B), which can perform their own designated functions. Example functions include convolutional, up-sampling, data transformation, pooling, and/or any other suitable functions. The output of the hidden layer (e.g., 604B) can then activate nodes of the next hidden layer (e.g., 604N), and so on. The output of the last hidden layer can activate one or more nodes of the output layer 606, at which point an output is provided. In some cases, while nodes (e.g., nodes 608A, 608B, 608C) in the neural network 610 are shown as having multiple output lines, a node has a single output and all lines shown as being output from a node represent the same output value.

In some cases, each node or interconnection between nodes can have a weight that is a set of parameters derived from training the neural network 610. For example, an interconnection between nodes can represent a piece of information learned about the interconnected nodes. The interconnection can have a numeric weight that can be tuned (e.g., based on a training dataset), allowing the neural network 610 to be adaptive to inputs and able to learn as more data is processed.

The neural network 610 can be pre-trained to process the features from the data in the input layer 602 using the different hidden layers 604 in order to provide the output through the output layer 606. In an example in which the neural network 610 is used to output text answers, the neural network 610 can be trained using training data that includes example question-answer pairs.

In some cases, the neural network 610 can adjust weights of nodes using a training process called backpropagation. Backpropagation can include a forward pass, a loss function, a backward pass, and a weight update. The forward pass, loss function, backward pass, and parameter update is performed for one training iteration. The process can be repeated for a certain number of iterations for each set of training media data until the weights of the layers are accurately tuned.

For example, the forward pass can include passing training data through the neural network 610. The weights can be initially randomized before the neural network 610 is trained. For a first training iteration for the neural network 610, the output can include values that do not give preference to any particular class due to the weights being randomly selected at initialization. For example, if the output is a vector with probabilities for different outputs, the probability value for each of the different outputs may be equal or at least very similar (e.g., for ten possible outputs, each output may have a probability value of 0.1). With the initial weights, the neural network 610 may be unable to determine low level features and thus may not make an accurate determination. A loss function can be used to analyze errors in the output. Any suitable loss function definition can be used.

The loss (or error) can be high for the first training dataset (e.g., images) since the actual values will be different than the predicted output. The goal of training is to minimize the amount of loss so that the predicted output comports with a target or ideal output. The neural network 610 can perform a backward pass by determining which inputs (weights) most contributed to the loss of the neural network 610, and can adjust the weights so that the loss decreases and is eventually minimized.

A derivative of the loss with respect to the weights can be computed to determine the weights that contributed most to the loss of the neural network 610. After the derivative is computed, a weight update can be performed by updating the weights of the filters. For example, the weights can be updated so that they change in the opposite direction of the gradient. A learning rate can be set to any suitable value, with a high learning rate including larger weight updates and a lower value indicating smaller weight updates.

The neural network 610 can include any suitable neural or deep learning network. One example includes a convolutional neural network (CNN), which includes an input layer and an output layer, with multiple hidden layers between the input and out layers. The hidden layers of a CNN can include a series of convolutional, nonlinear, pooling (for downsampling), and fully connected layers. In other examples, the neural network 610 can represent any other neural or deep learning network, such as a transformer network, an autoencoder, a deep belief nets (DBNs), a recurrent neural network (RNN), a large language model (LLM), etc.

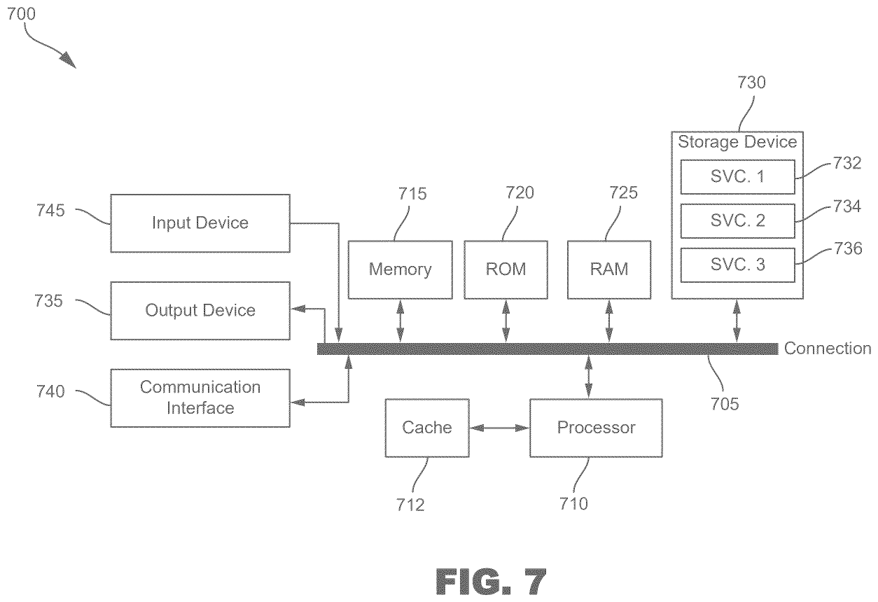

FIG. 7 illustrates an example computing device architecture 700 which can be employed to perform various steps, methods, and techniques disclosed herein. Specifically, the techniques described herein can be implemented, at least in part, through the computing device architecture 700 in an applicable computing device, such as logging tools 76. Further, the computing device can be configured to implement the techniques of analyzing colorimetric testing result(s) of drilling fluid as described herein (e.g., process 500, etc.). The various implementations will be apparent to those of ordinary skill in the art when practicing the present technology. Persons of ordinary skill in the art will also readily appreciate that other system implementations or examples are possible.

The components of the computing device architecture 700 are shown in electrical communication with each other using a connection 705, such as a bus. The example computing device architecture 700 includes a processing unit (CPU or processor) 710 and a computing device connection 705 that couples various computing device components including the computing device memory 715, such as read only memory (ROM) 720 and random-access memory (RAM) 725, to the processor 710.

The computing device architecture 700 can include a cache of high-speed memory connected directly with, in close proximity to, or integrated as part of the processor 710. The computing device architecture 700 can copy data from the memory 715 and/or the storage device 730 to the cache 712 for quick access by the processor 710. In this way, the cache can provide a performance boost that avoids processor 710 delays while waiting for data. These and other modules can control or be configured to control the processor 710 to perform various actions. Other computing device memory 715 may be available for use as well. The memory 715 can include multiple different types of memory with different performance characteristics. The processor 710 can include any general-purpose processor and a hardware or software service, such as service 1 732, service 2 734, and service 3 736 stored in storage device 730, configured to control the processor 710 as well as a special-purpose processor where software instructions are incorporated into the processor design. The processor 710 may be a self-contained system, containing multiple cores or processors, a bus, memory controller, cache, etc. A multi-core processor may be symmetric or asymmetric.

To enable user interaction with the computing device architecture 700, an input device 745 can represent any number of input mechanisms, such as a microphone for speech, a touch-sensitive screen for gesture or graphical input, keyboard, mouse, motion input, speech and so forth. An output device 735 can also be one or more of a number of output mechanisms known to those of skill in the art, such as a display, projector, television, speaker device, etc. In some instances, multimodal computing devices can enable a user to provide multiple types of input to communicate with the computing device architecture 700. The communications interface 740 can generally govern and manage the user input and computing device output. There is no restriction on operating on any particular hardware arrangement and therefore the basic features here may easily be substituted for improved hardware or firmware arrangements as they are developed.

Storage device 730 is a non-volatile memory and can be a hard disk or other types of computer readable media which can store data that are accessible by a computer, such as magnetic cassettes, flash memory cards, solid state memory devices, digital versatile disks, cartridges, random access memories (RAMs) 725, read only memory (ROM) 720, and hybrids thereof. The storage device 730 can include services 732, 734, 736 for controlling the processor 710. Other hardware or software modules are contemplated. The storage device 730 can be connected to the computing device connection 705. In one aspect, a hardware module that performs a particular function can include the software component stored in a computer-readable medium in connection with the necessary hardware components, such as the processor 710, connection 705, output device 735, and so forth, to carry out the function.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

Illustrative examples of the disclosure include:
Aspect 1. A system comprising: a camera configured to capture an image of a result of a colorimetric testing of an aqueous drilling fluid; a memory; and one or more processors coupled to the memory, the one or more processors being configured to: process, using a neural network, a collection of images captured by the camera to identify one or more color metric measurements; determine a chemical property of the aqueous drilling fluid based on the one or more color metric measurements; and in response to determining that the chemical property of the aqueous drilling fluid exceeds a threshold, determine a remedial action to adjust one or more parameters of a drilling fluid treatment schedule.
Aspect 2. The system of Aspect 1, wherein the colorimetric testing of the aqueous drilling fluid includes a methylene blue test (MBT) and the chemical property includes a reactivity of clays in the aqueous drilling fluid.
Aspect 3. The system of any of Aspects 1 to 2, wherein the one or more processors are further configured to: in response to determining that the chemical property of the aqueous drilling fluid exceeds the threshold, transmit a notification to a user, wherein the notification includes an analysis of the collection of images and the remedial action.
Aspect 4. The system of any of Aspects 1 to 3, wherein the one or more parameters of the drilling fluid treatment schedule, a rate of fluid treatment, a drilling fluid inhibition factor, a salt concentration of the aqueous drilling fluid, or a combination thereof.
Aspect 5. The system of any of Aspects 1 to 4, wherein processing the image of the result of the colorimetric testing of the aqueous drilling fluid includes analyzing one or more attributes associated with the one or more color metric measurements, wherein the one or more attributes include at least one of a color, a gradient, a shape, and a color change with respect to time.
Aspect 6. The system of any of Aspects 1 to 5, wherein the neural network includes a deep neural network comprising at least one of convolutional neural network, recurrent neural network, and generative neural network.
Aspect 7. The system of any of Aspects 1 to 6, wherein the one or more processors are further configured to: provide, to the neural network, data associated with at least one of light intensity, a density of the aqueous drilling fluid, and products used in the colorimetric testing.
Aspect 8. The system of any of Aspects 1 to 7, wherein the one or more processors are further configured to: in response to determining that the chemical property of the aqueous drilling fluid exceeds the threshold, generate an updated drilling fluid treatment schedule including the remedial action for a user.
Aspect 9. The system of any of Aspects 1 to 8, wherein the colorimetric testing includes an alkalinity testing, and the collection of images comprises continuous images captured over a period of time.
Aspect 10. The system of Aspect 9, wherein the chemical property of the aqueous drilling fluid includes at least one of mud alkalinity and filtrate alkalinity.
Aspect 11. A method comprising: receiving a collection of images capturing a result of a colorimetric testing of an aqueous drilling fluid; process, using a neural network, the collection of images to identify one or more color metric measurements; determine a chemical property of the aqueous drilling fluid based on the one or more color metric measurements; and in response to determining that the chemical property of the aqueous drilling fluid exceeds a threshold, determine a remedial action to adjust one or more parameters of a drilling fluid treatment schedule.
Aspect 12. The method of Aspect 11, wherein the colorimetric testing of the aqueous drilling fluid includes a methylene blue test (MBT) and the chemical property includes a reactivity of clays in the aqueous drilling fluid.
Aspect 13. The method of any of Aspects 11 to 12, further comprising: in response to determining that the chemical property of the aqueous drilling fluid exceeds the threshold, transmit a notification to a user, wherein the notification includes an analysis of the collection of images and the remedial action.
Aspect 14. The method of any of Aspects 11 to 13, wherein the one or more parameters of the drilling fluid treatment schedule, a rate of fluid treatment, a drilling fluid inhibition factor, a salt concentration of the aqueous drilling fluid, or a combination thereof.
Aspect 15. The method of any of Aspects 11 to 14, wherein processing the collection of images of the result of the colorimetric testing of the aqueous drilling fluid includes analyzing one or more attributes associated with the one or more color metric measurements, wherein the one or more attributes include at least one of a color, a gradient, a shape, and a color change with respect to time.
Aspect 16. The method of any of Aspects 11 to 15, wherein the neural network includes a deep neural network comprising at least one of convolutional neural network, recurrent neural network, and generative neural network.
Aspect 17. The method of any of Aspects 11 to 16, further comprising: provide, to the neural network, data associated with at least one of light intensity, a density of the aqueous drilling fluid, and products used in the colorimetric testing.
Aspect 18. The method of any of Aspects 11 to 17, further comprising: in response to determining that the chemical property of the aqueous drilling fluid exceeds the threshold, generate an updated drilling fluid treatment schedule including the remedial action for a user.
Aspect 19. The method of any of Aspects 11 to 18, wherein the colorimetric testing includes an alkalinity testing, and the collection of images comprises continuous images captured over a period of time.
Aspect 20. A non-transitory computer-readable medium having stored thereon instructions which, when executed by one or more processors, cause the one or more processors to perform a method according to any of Aspects 11 to 19.
Aspect 21. A system comprising means for performing a method according to any of Aspects 11 to 19.
Aspect 22. A computer-program product having stored thereon instructions which, when executed by one or more processors, cause the one or more processors to perform a method according to any of Aspects 11 to 19.

What is claimed is:

1. A system comprising:
a camera configured to capture an image of a result of a colorimetric testing of an aqueous drilling fluid;
a memory; and
one or more processors coupled to the memory, the one or more processors being configured to:
process, using a neural network, a collection of images captured by the camera to identify one or more color metric measurements;
determine a chemical property of the aqueous drilling fluid based on the one or more color metric measurements; and
in response to determining that the chemical property of the aqueous drilling fluid exceeds a threshold, determine a remedial action to adjust one or more parameters of a drilling fluid treatment schedule.

2. The system of claim 1, wherein the colorimetric testing of the aqueous drilling fluid includes a methylene blue test (MBT) and the chemical property includes a reactivity of clays in the aqueous drilling fluid.

3. The system of claim 1, wherein the one or more processors are further configured to:
in response to determining that the chemical property of the aqueous drilling fluid exceeds the threshold, transmit a notification to a user, wherein the notification includes an analysis of the collection of images and the remedial action.

4. The system of claim 1, wherein the one or more parameters of the drilling fluid treatment schedule, a rate of fluid treatment, a drilling fluid inhibition factor, a salt concentration of the aqueous drilling fluid, or a combination thereof.

5. The system of claim 1, wherein processing the image of the result of the colorimetric testing of the aqueous drilling fluid includes analyzing one or more attributes associated with the one or more color metric measurements, wherein the one or more attributes include at least one of a color, a gradient, a shape, and a color change with respect to time.

6. The system of claim 1, wherein the neural network includes a deep neural network comprising at least one of convolutional neural network, recurrent neural network, and generative neural network.

7. The system of claim 1, wherein the one or more processors are further configured to:
provide, to the neural network, data associated with at least one of light intensity, a density of the aqueous drilling fluid, and products used in the colorimetric testing.

8. The system of claim 1, wherein the one or more processors are further configured to:
in response to determining that the chemical property of the aqueous drilling fluid exceeds the threshold, generate an updated drilling fluid treatment schedule including the remedial action for a user.

9. The system of claim 1, wherein the colorimetric testing includes an alkalinity testing, and the collection of images comprises continuous images captured over a period of time.

10. The system of claim 9, wherein the chemical property of the aqueous drilling fluid includes at least one of mud alkalinity and filtrate alkalinity.

11. A method comprising:
receiving a collection of images capturing a result of a colorimetric testing of an aqueous drilling fluid;
processing, using a neural network, the collection of images to identify one or more color metric measurements;
determining a chemical property of the aqueous drilling fluid based on the one or more color metric measurements; and
in response to determining that the chemical property of the aqueous drilling fluid exceeds a threshold, determining a remedial action to adjust one or more parameters of a drilling fluid treatment schedule.

12. The method of claim 11, wherein the colorimetric testing of the aqueous drilling fluid includes a methylene blue test (MBT) and the chemical property includes a reactivity of clays in the aqueous drilling fluid.

13. The method of claim 11, further comprising:
in response to determining that the chemical property of the aqueous drilling fluid exceeds the threshold, transmitting a notification to a user, wherein the notification includes an analysis of the collection of images and the remedial action.

14. The method of claim 11, wherein the one or more parameters of the drilling fluid treatment schedule, a rate of fluid treatment, a drilling fluid inhibition factor, a salt concentration of the aqueous drilling fluid, or a combination thereof.

15. The method of claim 11, wherein processing the collection of images of the result of the colorimetric testing of the aqueous drilling fluid includes analyzing one or more attributes associated with the one or more color metric measurements, wherein the one or more attributes include at least one of a color, a gradient, a shape, and a color change with respect to time.

16. The method of claim 11, wherein the neural network includes a deep neural network comprising at least one of convolutional neural network, recurrent neural network, and generative neural network.

17. The method of claim 11, further comprising:
providing, to the neural network, data associated with at least one of light intensity, a density of the aqueous drilling fluid, and products used in the colorimetric testing.

18. The method of claim 11, further comprising:
in response to determining that the chemical property of the aqueous drilling fluid exceeds the threshold, generating an updated drilling fluid treatment schedule including the remedial action for a user.

19. The method of claim 11, wherein the colorimetric testing includes an alkalinity testing, and the collection of images comprises continuous images captured over a period of time.

20. A non-transitory computer-readable medium having stored thereon instructions which, when executed by one or more processors, cause the one or more processors to:
receive a collection of images capturing a result of a colorimetric testing of an aqueous drilling fluid;
process, using a neural network, the collection of images to identify one or more color metric measurements;
determine a chemical property of the aqueous drilling fluid based on the one or more color metric measurements; and
in response to determining that the chemical property of the aqueous drilling fluid exceeds a threshold, determine a remedial action to adjust one or more parameters of a drilling fluid treatment schedule.
